(19) United States
(12) Patent Application Publication
(10) Pub. No.: US 2014/0271884 A1
(43) Pub. Date: Sep. 18, 2014

(54) HIGH-LOADING NANOPARTICLE-BASED FORMULATION FOR WATER-INSOLUBLE STEROIDS

(71) Applicant: The Trustees of Princeton University, Princeton, NJ (US)

(72) Inventors: Robert K. Prud'homme, Lawrenceville, NJ (US); Carlos E. Figueroa, Maplewood, NJ (US)

(21) Appl. No.: 14/352,576

(22) PCT Filed: Oct. 25, 2012
(86) PCT No.: PCT/US12/61945

§ 371 Date: Apr. 17, 2014

Related U.S. Application Data
(60) Provisional application No. 61/551,221, filed on Oct. 25, 2011.
(60) Provisional application No. 61/562,763, filed on Nov. 22, 2011.

(51) Int. Cl. A61K 9/14; A61K 31/57

(52) U.S. Cl. CPC A61K 9/146; A61K 31/57
USPC 424/489; 514/177

(57) ABSTRACT

Compositions comprising water-insoluble steroids encapsulated into nanoparticle forms are provided. Methods of preparing water-insoluble steroids encapsulated in a nanoparticle form are provided. Methods of treating disease by delivering a water-insoluble steroid encapsulated into nanoparticle form to a patient in need thereof are provided.

This application claims the benefit of U.S. Provisional Application No. 61/551,221, filed Oct. 25, 2011, and U.S. Provisional Application No. 61/562,763, filed Nov. 22, 2011, both of which are incorporated herein by reference as if fully set forth.

This invention was made with government support under Grant #CTS-0506966 awarded by the National Science Foundation. The government has certain rights in this invention.

### FIELD OF INVENTION

The disclosure relates to water-insoluble steroids encapsulated in nanoparticle forms.

### BACKGROUND

Most nanoparticle formulations of water-insoluble steroids cannot achieve high weight loadings of the active in the nanoparticulate construct. While there are polymeric micellar formulations, such as that described by Soo et al. for estradiol, that are able to attain weight loadings of steroid of up to 66%, these formulations are largely dependent on the partition coefficients and the crystallization kinetics of the steroid of interest. When attempting to formulate steroids that are very insoluble in water, have relatively low affinity for hydrophobic entities, and rapidly crystallize when exposed to aqueous systems, delivery systems are generally limited. For steroids with reactive terminal groups, such as prednisone, one possible method for incorporating the active into a lipophilic vehicle is to attach a hydrophobic moiety through a cleavable linkage (A. D. Woolfson et al.). By making the compound more hydrophobic, it is possible to better encapsulate the steroid within a hydrophobic environment of polymeric micelles or nanoparticles.

However, this approach is not possible in the case of steroids with no reactive end groups. In the case of progesterone, the literature has reported several types of formulations. For example, crystalline nanosuspensions stabilized by stearic acid have been reported at progesterone concentrations of less than 2.7 mg/mL (Salem). Both Cavalli et al. and Duchêne et al. describe the complexation of the active with 2-hydroxypropyl-β-cyclodextrin in order to increase the water solubility of progesterone. They utilize this to produce solid lipid nanoparticles (SLN) with drug loadings of up to 2 wt %. However, Memisoglu and colleagues have made nanospheres, using progesterone-β-cyclodextrincomplexes, with drug loadings less than 12 wt %. In addition, polymeric nanoparticles have been made with drug loadings of less than 10 wt % (Matsumoto et al.). Nevertheless, none of these formulations are reported at progesterone concentrations of 2.7 mg/mL or higher.

### SUMMARY

In an aspect, the invention relates to a composition comprising an amphiphilic polymer and one or more water-insoluble steroids with a partition coefficient of log P>3.0. The amphiphilic polymer is in a nanoparticle form and the one or more water insoluble steroids are encapsulated in the nanoparticle form. The nanoparticle form is dispersable in an aqueous medium.

In an aspect, the invention relates to a method of preparing a water-insoluble steroid encapsulated in a nanoparticle form. The method includes dissolving at least one amphiphilic polymer and at least one water-insoluble steroid in an organic solvent to form an organic solvent stream. The method also includes rapidly mixing the organic solvent stream with an aqueous stream.

In an aspect, the invention relates to a composition made by a method of preparing a water-insoluble steroid encapsulated in a nanoparticle form. The method includes dissolving at least one amphiphilic polymer and at least one water-insoluble steroid in an organic solvent to form an organic solvent stream. The method also includes rapidly mixing the organic solvent stream with an aqueous stream.

In an aspect the invention relates to a method of treating disease. The method includes delivering to a patient in need thereof a composition comprising an amphiphilic polymer and one or more water-insoluble steroids with a partition coefficient of log P>3.0. The amphiphilic polymer is in a nanoparticle form and the one or more water insoluble steroids are encapsulated in the nanoparticle form. The nanoparticle form is dispersable in an aqueous medium.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 illustrates particle diameter of progesterone loaded with cholesterol into PEG-b-PLA nanoparticles.

FIG. 2 illustrates particle diameter of progesterone loaded with prednisone diglycolate dimer into PEG-b-PLA nanoparticles.

FIG. 3 illustrates particle diameter of progesterone loaded with prednisone cosanyl diglycolate into PEG-b-PLA nanoparticles.

FIG. 4A illustrates particle diameter of progesterone loaded with α-tocopherol into PEG-b-PLA nanoparticles.

FIG. 4B illustrates particle diameter of progesterone loaded with α-tocopherol into PEG-b-PLA nanoparticles.

FIG. 5 illustrates particle diameter of lyophilized and redispersed progesterone-tocopherol co-loaded PEG-b-PLA nanoparticles.

FIG. 6A illustrates UV/visible absorbance measurements of progesterone in THF.

FIG. 6B illustrates the peak of progesterone in solution.

FIG. 7A illustrates particle diameter of concentrated progesterone-tocopherol co-loaded PEG-b-PLA nanoparticles.

FIG. 7B illustrates particle diameter growth.

FIG. 8A illustrates particle diameter of trial progesterone-loaded nanoparticles.

FIG. 8B illustrates α-tocopherol concentration progesterone encapsulation efficiency.

FIG. 9A illustrates particle diameter of trial progesterone-loaded nanoparticles.

FIG. 9B illustrates nanoparticle component concentration progesterone encapsulation efficiency.

FIG. 10 illustrates progesterone levels in rat plasma after nanoparticle delivery.

### DETAILED DESCRIPTION OF THE PREFERRED EMBODIMENTS

Certain terminology is used in the following description for convenience only and is not limiting. The words “right,” “left,” “top,” and “bottom” designate directions in the drawings to which reference is made. The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

Embodiments include one or more water-insoluble steroid encapsulated in a nanoparticle form. Embodiments include a composition comprising one or more water-insoluble steroid with a partition coefficient of log P>3.0 encapsulated into a nanoparticle form dispersable in an aqueous medium. A water-insoluble steroid of the one or more water-insoluble steroids may be an active steroid. The nanoparticle form includes an amphiphilic polymer. The amphiphilic polymer may be referred to as a stabilizing amphiphilic polymer as it provides a steric stablilizing layer on the nanoparticle surface. A stablizing amphiphilic polymer may coat the outside of a nanoparticle and prevent its aggregation and growth. The nanoparticle form may be in a size range between and including any value from 40 to 1000 nm. The nanoparticle form may be in a size range between and including any value from 50 to 1000 nm. The nanoparticle form may be in a size range between and including any value from 80 to 350 nm. The nanoparticle form may have a size in a range between and including any two integer values from 40 to 1000 nm. The nanoparticle form may have a size in a range between and including any two integer values from 50 to 1000 nm. The composition may be a drug-delivery vehicle for water-insoluble steroids. The nanoparticle encapsulation may be effected by processing the core components with an amphiphilic polymeric stabilizer in a rapid micromixing process described as Flash NanoPrecipitation.

The amphiphilic polymer, also referred to as a stabilizing amphiphilic polymer, may be an amphiphilic copolymer. The amphiphilic polymer may be a copolymer of a hydrophilic block coupled with a hydrophobic block.

Hydrophobic blocks may include but are not limited to acrylates, methacrylates, acrylonitriles, methacrylonitrile, vinyls, aminoalkyls, styrenes, cellulose acetate phthalate, cellulose acetate succinate, hydroxypropylmethylcellulose phthalate, poly(D,L lactide), poly(D,L-lactide-co-glycolide), poly(D,L caprolactam), poly(D,L caprolactone), poly(glycolide), poly(hydroxybutyrate), poly(alkylcarbonate), poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Illum, L., Davids, S. S. (eds.) Polymers in Controlled Drug Delivery Wright, Bristol, 1987; Arshady, J. Controlled Release 17:1-22, 1991; Pitt, Int. J. Phar. 59:173-196, 1990; Holland et al., J. Controlled Release 4:155-0180, 1986), hydrophobic peptide-based polymers, copolymers based on poly(L-amino acids) (Lavasanifar, A., it al., Advanced Drug Delivery Reviews (2002) 54:169-190), poly(ethylene-vinyl acetate) (“EVA”) copolymers, silicone rubber, polyethylene, polypropylene, polydienes, maleic anyhydride copolymers of vinyl methylether and other vinyl ethers, polyamides (nylon 6,6), polyurethane, poly(ester urethanes), poly(ether urethanes), poly(ester-urea). Acrylates may include methyl acrylate, ethyl acrylate, propyl acrylate, n-butyl acrylate (BA), isobutyl acrylate, 2-ethyl acrylate, and t-butyl acrylate. Methacrylates may include ethyl methacrylate, n-butyl methacrylate, and isobutyl methacrylate. Vinyls may include vinyl acetate, vinylversatate, vinylpropionate, vinylformamide, vinylacetamide, vinylpyridines, and vinyllimidazole. Aminoaklyls may include aminoalkylacrylates, aminoalkylsmethacrylates, and aminoalkyl(meth)acrylamides. Polydienes may include polybutadiene, polyisoprene and hydrogenated forms of these polymers.

Hydrophobic blocks also include but are not limited to poly(ethylenevinyl acetate), poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly(caprolactone) or poly(lactic acid) For non-biologically related applications hydrophobic blocks may include but are not limited to polystyrene, polyacrylates, and butadienes.

Natural products with sufficient hydrophobicity to act as the hydrophobic portion of the amphiphilic polymer may include but are not limited to hydrophobic vitamins, carotenoids, retinols, cholecalciferol, calcitriol, hydroxycholecalciferol, ergocalciferol, alpha-tocopherol, alpha-tocopherol acetate, alpha-tocopherol nicotinate, and estradiol. Hydrophobic vitamins may include vitamin E, vitamin K, and vitamin A. Retinols may include beta carotene, astazanthin, trans and cis retinal, retinoic acid, folic acid, dihydrofolate, retinylacetate, and retinyl palmintate. Hydrophobic blocks may be vitamin E, which can be readily obtained as a vitamin E succinate, which may facilitate functionalization to amines and hydroxyls on the active species.

Hydrophilic blocks may include but are not limited to carboxylic acids, polyoxyethylenes, poly ethylene oxide, polyacrylamides and copolymers thereof with dimethylaminoethylmethacrylate, diallyldimethylammonium chloride, vinylbenzylthrimethylammonium chloride, acrylic acid, methacrylic acid, 2-crrylamideo-2-methylpropane sulfonic acid and styrene sulfonate, polyvincyl pyrrolidone, starches and starch derivatives, dextran and dextran derivatives, polypeptides, poly hyaluronic acids, alginic acids, polylactides, polyethyleneimines, polyionenes, polyacrylic acids, and polyiminocarboxylates, gelatin, and unsaturated ethylenic mono or dicarboxylic acids.

Carboxylic acids may include but are not limited to acrylic acid, methacrylic acid, itaconic acid, and maleic acid. Polypeptides may include polylysines, polyarginines, and polyglutamic acids.

Nanoparticles may be formed with graft, block or random amphiphilic copolymers. These copolymers may have a molecular weight between 1000 g/mole and 50,000 g/mole, between about 3000 g/mole to about 25,000 g/mole, or at least 2000 g/mole. These copolymers may have a molecular weight having a value in a range between and including any two integer values from 1000 g/mole to 50,000 g/mole. The amphiphilic copolymers used may exhibit a water surface tension of at least 50 dynes/cm² at a concentration of 0.1 wt %.

The blocks may be diblock or triblock repeats. Block copolymers may include blocks of polystyrene, polyethylene, polybutyl acrylate, polybutyl methacrylate, polylactic acid, polycaprolactone, polyacrylic acid, polyoxyethylene and polyacrylamide. A listing of hydrophilic polymers that may be included in the amphiphilic polymer can be found in Handbook of Water-Soluble Gums and Resins, R. Davidson, McGraw-Hill (1980), which is incorporated herein by reference as if fully set forth.

In graft copolymers, the length of a grafted moiety may vary. The grafted segments may be alkyl chains of 4 to 18 carbons or equivalent to 2 to 9 ethylene units in length. In addition, the grafting of the polymer backbone may be useful to enhance solvation or nanoparticle stabilization properties. A grafted butyl group on the hydrophobic backbone of a diblock copolymer of a polyethylene and polyethylene glycol may increase the solubility of the polyethylene block. Chemical moieties grafted to the block unit of the copolymer may comprise alkyl chains containing species such as amides, imides, phenyl, carboxy, aldehyde or alcohol groups.

A water insoluble steroid may be present in the nanoparticles at any concentration. A water insoluble steroid may be present in the nanoparticles at a concentration of 12.5% by weight or greater, where the percent is the weight of steroid divided by the total weight of nanoparticle. A water insoluble steroid may be present in the nanoparticles at a concentration of 12.5%-60% by weight. A water insoluble steroid may be present in the nanoparticles at a concentration in a range between and including any two integer values from 13%-60% by weight. A water insoluble steroid may be present in the nanoparticles at a concentration of 45% by weight. The one or more water insoluble steroid may include but is not limited to at least one substance selected from the group of cholesterol, β-sitosterol, campesterol, ergosterol, cholecalciferol, fusidic acid, lanosterol, stigmasterol, cholestane, cyproterone, danazol, dydrogesterone, megestrol, canrenone, medrogestone, ethisterone, spironolactone, testosterone, estradiol, pregnenolone, 17-hydroxypregnenolone, progesterone, 17-hydroxyprogesterone, androstenedione, androsterone, epiandrosterone, dehydroepiandrosterone, epitestosterone, dihydrotestosterone, estrone, deoxycorticosterone, tixocortol pivalate, mometasone, aclometasone, betamethasone valerate, prednicarbate, clobetasol, ciclesonide, rimexolone, clobetasone, halobetasol propionate, and a derivative of each of the foregoing. The derivative may be but is not limited to a salt or a pharmaceutically acceptable salt. The derivatives may be chemical modifications of the steroids with a group including but not limited to esters, fluorine, methoxy, ethyl, butyl, propyl, hexyl, or other organic moieties. The one or more water insoluble steroid may also include but is not limited to at least one substance selected from the group of other steroid derivatives and conjugated prodrugs.

In an embodiment, aqueous dispersions of the nanoparticles may be produced in an injectable form with steroid concentrations of 3 mg/ml to 200 mg/ml of active steroid. The steroid concentration in the aqueous dispersion may be a value in a range between and including any two integers from 3 mg/ml to 200 mg/ml.

The composition may include one or more water-insoluble co-solute with a partition coefficient of log P>3.0. The composition may include one or more hydrophobic co-solute with a partition coefficient of log P>2. The one or more water-insoluble co-solute may include but is not limited to at least one agent selected from the group of hydrophobic vitamins, carotenoids, retinols, cholecalciferol, calcitriol, hydroxycholecalciferol, ergocalciferol, α-tocopherol, α-tocopherol acetate, α-tocopherol nicotinate, estradiol, lipids and a derivative of each of the forgoing. The derivative may be but is not limited to a salt or a pharmaceutically acceptable salt. The derivative may be a chemical modification of the steroid with a group including but not limited to esters, fluorine, methoxy, ethyl, butyl, propyl, hexyl, or other organic moieties. Examples of hydrophobic vitamins include but are not limited to vitamin E, vitamins K and A. Carotenoids and retinols may include but are not limited to beta carotene, astaxanthin, trans and cis retinal, retinoic acid, folic acid, dihydrofolate, retinyl acetate, and retinyl palmitate.

The composition may include a pharmaceutically acceptable carrier or an excipient. These components may be present to aid in the processing, stability, or delivery of the active ingredients. Pharmaceutically acceptable carriers or excipients that may be a part of a composition herein include but are not limited to at least one of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.), and phosphate buffered saline (PBS). The substance(s) or agent(s) in a composition herein may be in the form of a pharmaceutically acceptable salt. Pharmaceutically acceptable salts that may be included in embodiments herein can be found in Handbook of Pharmaceutical Salts: Properties, Selection, and Use, Stahl and Wermuth (Eds.), VHCA, Verlag Helvetica Chimica Acta (Zurich, Switzerland) and WILEY-VCH (Weinheim, Federal Republic of Germany); ISBN: 3-906390-26-8, which is incorporated herein by reference as if fully set forth.

Embodiments include a method of preparing a water-insoluble steroid encapsulated in a nanoparticle form. The method may include dissolving at least one amphiphilic polymer and one or more water-insoluble steroid in an organic solvent to form an organic solvent stream, and Flash NanoPrecipitation by high intensity mixing of the organic solvent stream with an aqueous stream. The one or more water-insoluble steroid may have a partition coefficient of log P>3.0. High intensity mixing, which is also referred to as rapid mixing herein, means that the solvent interdiffusion between the water-misciple organic solvent and the aqueous solvent happens on a time frame more rapid than the time for precipitation and growth of the nanoparticle. The condition of rapid mixing for Flash NanoPrecipitation is met when the final nanoparticle size no longer significantly decreases upon application of more rapid mixing. Rapid mixing may be effected by collision of two or more liquid streams in a confined cavity such as in a Confined Impinging Jet (CIJ) mixer or a multi-inlet vortex (MIVM) mixer, or it may be effected by the rapid injection of the organic stream into an aqueous liquid volume which is itself being stirred. Examples of the lack of nanoparticle size upon mixing rate in this regime of rapid mixing are shown in Johnson and Prud'homme (2003), Liu et al. (2007) and Johnson (2003), which are incorporated herein by reference as if fully set forth.

In an embodiment, the ratio of water insoluble steroid to amphiphilic polymer may be 1:0.1 up to 1:10, and in the organic stream the water insoluble steroid may be at 0.01 mg/ml up to 200 mg/ml. The water insoluble steroid may be at 1 mg/ml up to 20 mg/ml in the organic stream and the ratio of water insoluble steroid to amphiphilic polymer may be 1:0.1 up to 1:10. The water insoluble steroid may be at concentration in a range between and including any two values in 0.01 mg/ml steps from 0.01 mg/ml up to 200 mg/ml in the organic stream and the ratio of water insoluble steroid to amphiphilic polymer may be 1:0.1 up to 1:10.

The process of Flash NanoPrecipitation is briefly described as follows. Flash NanoPrecipitation (FNP), is a rapid solvent displacement precipitation that uses amphiphilic copolymers to direct self-assembly of nanoparticles. Particle formation by FNP may involve two primary steps: (i) dissolution of the carrier/stabilizer (e.g., amphiphilic copolymer) and organic active(s) (e.g. hydrophobic drugs, or one or more water insoluble steroid with or without one or more water-insoluble co-solute) into a aqueous-miscible, organic solvent and (ii) rapid mixing of the obtained solution with an anti-solvent (aqueous phase) to create high supersaturation of the components.

Without being limited to a particular mechanism of action, it is believed that the process relies on the interplay of multiple timescales, namely (i) time for homogeneous mixing of streams (τₘᵢₓ), (ii) time for nucleation and growth of soluble organic compound(s) (τ_{ng}), and (iii) time for precipitation and self-assembly of the amphiphilic copolymer stabilizer (τₛₐ). When τₘᵢₓ is sufficiently fast, such that a selective change in solvent quality is very rapidly induced, the mixture becomes supersaturated in all hydrophobic components. Under these conditions, nucleation and growth of the solute proceed in the diffusion-limited regime. By balancing the nucleation and growth times of the active(s) with the block copolymer assembly time (τₘᵢₓ<<τₛₐ≅τ_{ng}), it is possible to block nuclei growth by adsorption of polymer chains on growing particle surfaces, resulting in sub-micron particle sizes and narrow particle size distributions.

Since particle formation is extremely rapid, mixing should be very fast. Millisecond (c.a. 10 ms) mixing has been demonstrated in confined geometries (Lui et al.). Mixing may be for a time of 1-100 ms. Mixing may be for a time less than 1 ms. Mixing may be for a time in a range between any two integer values from 1-100 ms.

Briefly, the nanoparticles are formed in a precipitation technique where an organic solvent stream containing the dissolved steroid and an amphiphilic polymer, as well as a co-solute, if desired, is rapidly mixed against an aqueous stream. In the case that a steroid does not encapsulate well within the polymer shell, a co-solute may used. For the case of progesterone, which may not be derivatized to be more hydrophobic, the resulting nanoparticles can have steroid encapsulation rates of up to 70% with nanoparticle loadings of up to 25 wt % and can be made such that the concentration of suspended steroid in the aqueous mixture is up to 10 mg/mL. The method may include lyophilizing the nanoparticles after formation. The step of lyophilizing may be conducted quickly. The method may include adding cryoprotectants before or during the step of lyophilizing. Lyophilizing may be beneficial in the cases where the nanoparticles are not stable for too long. The method may include resuspending lyophilized nanoparticles.

The one or more water insoluble steroid dissolved in the organic solvent may include but is not limited to at least one substance selected from the group of cholesterol, β-sitosterol, campesterol, ergosterol, cholecalciferol, fusidic acid, lanosterol, stigmasterol, cholestane, cyproterone, danazol, dydrogesterone, megestrol, canrenone, medrogestone, ethisterone, spironolactone, testosterone, estradiol, pregnenolone, 17-hydroxypregnenolone, progesterone, 17-hydroxyprogesterone, androstenedione, androsterone, epiandrosterone, dehydroepiandrosterone, epitestosterone, dihydrotestosterone, estrone, deoxycorticosterone, tixocortol pivalate, mometasone, aclometasone, betamethasone valerate, prednicarbate, clobetasol, ciclesonide, rimexolone, clobetasone, halobetasol propionate, and a derivative each of the foregoing. The one or more water insoluble steroid dissolved in the organic solvent may also include but is not limited to at least one substance selected from the group of other steroid derivatives and conjugated prodrugs.

The organic solvent may include a mixture of solvents or a single organic solvent. The organic solvent may be an alcohol or an ether. The organic solvent may be methanol. The organic solvent may be tetrahydrofuran (“THF”). The organic solvent may be dimethylsulfoxide (DMSO), dimethylformamide (DMF), or n-methylpyrollidone (NMP). The organic solvent may be any organic solvent that is miscible with the aqueous phase at a concentration greater than 10% wt and into which the water-insoluble steroids, co-solutes and amphiphilic polymer is soluble up to 0.1 wt %. Temperature may be used to increase the organic solvent's miscibility with the aqueous phase and the solubility of the actives.

The aqueous stream may include pure deionized water, or it may contain other excipients. These excipients may include buffers to control pH, salts to control solubility and precipitation kinetics, or other excipients such as listed above which may provide aids for post-processing or delivery of the formulation. Among the uses of these excipients would be antibacterial agents for sterility, cryoprotectants and lyoprotectants to aid in lyophilization of the nanoparticles. A composition herein may include one or more of these agents.

The method may include dissolving one or more water-insoluble co-solute along with the amphiphilic polymer and the one or more water-insoluble steroid in the organic solvent. The one or more water-insoluble co-solute may have a partition coefficient of log P>3.0. The one or more hydrophobic co-solute may have a partition coefficient of log P>2. The one or more water-insoluble co-solute dissolved in the organic solvent may include but is not limited to at least one agent selected from the group of hydrophobic vitamins, carotenoids, retinols, cholecalciferol, calcitriol, hydroxycholecalciferol, ergocalciferol, α-tocopherol, α-tocopherol acetate, α-tocopherol nicotinate, estradiol, and a derivative of each of the forgoing. Examples of hydrophobic vitamins include but are not limited to vitamin E, vitamins K and A. Carotenoids and retinols may include but are not limited to beta carotene, astaxanthin, trans and cis retinal, retinoic acid, folic acid, dihydrofolate, retinyl acetate, and retinyl palmitate.

Flash NanoPrecipitation (FNP) may use one of several geometries to effect rapid micromixing. These may include but are not limited to an MIVM mixer or a CIJ mixer. A method herein may implement any of the several geometries to effect rapid mixing. A method herein may be implemented with a MIVM or CIJ mixer to effect rapid mixing.

Rapid mixing may require an intense enough mixing velocity so that solvent displacement (i.e. interdiffusion of the organic and aqueous solvents to achieve a uniform composition) occurs more rapidly than particle growth or stabilizing polymer assembly, as described above. The appropriate mixing intensity for rapid mixing can be determined in a simple set of experiments, without undue experimentation, by increasing mixing velocity or intensity until no further reduction in nanoparticle size is observed. The value of the mixing intensity will depend on the geometry used, the concentration and supersaturation of the actives and co-solutes. For the CIJ geometry the stream velocities required for nanoparticle formation using beta-carotene as a model active have been presented by Liu et al., and Johnson and Prud'homme, which are incorporated herein by reference as if fully set forth. For the MIVM examples of mixing intensities for effect nanoparticle formation have been presented by Liu et al., which is incorporated herein by reference as if fully set forth. The velocity of the fluid streams create the mixing in the MIVM and CIJ. Increasing the velocity of the fluid streams increases the mixing intensity.

The method may allow production of nanoparticles loaded with a relatively large amount of water-insoluble steroids. Although most steroids are not water-soluble, they are also generally not well encapsulated in nanoparticles. Thus, embodiments herein present a novel method of loading water-insoluble steroids within an amphiphilic block copolymer shell through the aid of a co-solute. Through the use of Flash NanoPrecipitation to make the nanoparticles, up to 24 wt % loading of steroids was achieved. This large loading can be useful in delivering the actives for the treatment of various diseases, such as cancer, inflammatory disease, and traumatic brain injury.

An embodiment includes a composition made by any method herein.

An embodiment includes a method of treating disease. The method may include delivering any composition herein to a patient in need thereof. The composition may include one or more water-insoluble steroid with a partition coefficient of log P>3.0 encapsulated into a nanoparticle form dispersable in an aqueous medium. A water-insoluble steroid of the one or more water-insoluble steroids may be an active steroid. The nanoparticle form may be in a size range between and including any value from 50 to 1000 nm. The nanoparticle form may be in a size range between and including any value from 80 to 350 nm. The nanoparticle form may have a size in a range between and including any two integer values from 50 to 1000 nm. The composition may be a drug-delivery vehicle for water-insoluble steroids. The disease may be but is not limited to inflammatory disease, cancer and traumatic brain injury. The patient in need thereof may suffer from at least one disease selected from but not limited to inflammatory disease, cancer or traumatic brain injury.

Delivering may be by way of any route including but not limited to at least one of oral, injection, topical, enteral, rectal, gastrointestinal, sublingual, sublabial, buccal, epidural, intracerebral, intracerebroventricular, intracisternal, epicutaneous, intradermal, subcutaneous, nasal, intravenous, intraarterial, intramuscular, intracardiac, intraosseous, intrathecal, intraperitoneal, intravesical, intravitreal, intracavernous, intravaginal, intrauterine, extra-amniotic, transdermal, intratumoral, or transmucosal.

The one or more water insoluble steroid may include but is not limited to at least one substance selected from the group of cholesterol, β-sitosterol, campesterol, ergosterol, cholecalciferol, fusidic acid, lanosterol, stigmasterol, cholestane, cyproterone, danazol, dydrogesterone, megestrol, canrenone, medrogestone, ethisterone, spironolactone, testosterone, estradiol, pregnenolone, 17-hydroxypregnenolone, progesterone, 17-hydroxyprogesterone, androstenedione, androsterone, epiandrosterone, dehydroepiandrosterone, epitestosterone, dihydrotestosterone, estrone, deoxycorticosterone, tixocortol pivalate, mometasone, aclometasone, betamethasone valerate, prednicarbate, clobetasol, ciclesonide, rimexolone, clobetasone, halobetasol propionate, and a derivative each of the foregoing. The one or more water insoluble steroid may also include but is not limited to at least one substance selected from the group of other steroid derivatives and conjugated prodrugs.

The composition may include one or more water-insoluble co-solute with a partition coefficient of log P>3.0. The one or more water-insoluble co-solute may include but is not limited to at least one agent selected from the group of hydrophobic vitamins, carotenoids, retinols, cholecalciferol, calcitriol, hydroxycholecalciferol, ergocalciferol, α-tocopherol, α-tocopherol acetate, α-tocopherol nicotinate, estradiol, and a derivative of each of the forgoing. Examples of hydrophobic vitamins include but are not limited to vitamin E, vitamins K and A. Carotenoids and retinols may include but are not limited to beta carotene, astaxanthin, trans and cis retinal, retinoic acid, folic acid, dihydrofolate, retinyl acetate, and retinyl palmitate.

Since high loadings of steroids can be achieved in nanoparticles composed of safe FDA-approved components, the compositions herein when provided as a drug-delivery vehicle could be a useful substitute for parenteral application. Other steroid nanoparticle formulations generally have a low loading of the active and thus are not feasible for injection; however, by applying nanoparticles that have close to 30 wt % loadings of the steroids, it is possible to deliver an effective concentration in a reasonable injection volume.

Embodiments contained herein can achieve high drug loadings of up to 25 wt % and can reach high concentrations in aqueous suspension with relative stability. Through such high loadings, it is feasible to reach high concentrations of the active agent in small volumes. Furthermore, since these nanoparticles can be formulated using FDA-approved biocompatible amphiphilic polymers and co-solutes, the use of toxic excipients (i.e., β-cyclodextrin) is no longer necessary to formulate other vehicles for the steroids. This is advantageous for the administration of steroids to patients who suffer from many diseases, such as rheumatoid arthritis, many types of cancer, and traumatic brain injury.

### Embodiments

The following list includes particular embodiments of the present invention. The list, however, is not limiting and does not exclude the above embodiments, the below examples, or alternate embodiments, as would be appreciated by one of ordinary skill in the art.

1. A composition comprising an amphiphilic polymer and one or more water-insoluble steroids with a partition coefficient of log P>3.0, wherein the amphiphilic polymer is in a nanoparticle form and the one or more water insoluble steroids are encapsulated in the nanoparticle form, and the nanoparticle form is dispersable in an aqueous medium.

2. The composition of embodiment 1, wherein the nanoparticle form is 40 to 1000 nm.

3. The composition of embodiment 1, wherein the nanoparticle form is 80 to 350 nm.

4. The composition of any one or more of embodiments 1-3, wherein the one or more water-insoluble steroids are selected from the group consisting of cholesterol; β-sitosterol; campesterol; ergosterol; cholecalciferol; fusidic acid; lanosterol; stigmasterol; cholestane; cyproterone; danazol; dydrogesterone; megestrol; canrenone; medrogestone; ethisterone; spironolactone; testosterone; estradiol; pregnenolone; 17-hydroxypregnenolone; progesterone; 17-hydroxyprogesterone; androstenedione; androsterone; epiandrosterone; dehydroepiandrosterone; epitestosterone; dihydrotestosterone; estrone; deoxycorticosterone; tixocortol pivalate; mometasone; aclometasone; betamethasone valerate; prednicarbate; clobetasol; ciclesonide; rimexolone; clobetasone; and halobetasol propionate; or a derivative of any of the foregoing.

5. The composition of any one or more of embodiments 1-4, wherein the amphiphilic polymer is a graft amphiphilic polymer, a block amphiphilic polymer, or a random amphiphilic polymer.

6. The composition of any one or more of embodiments 1-5, wherein the amphiphilic polymer has a molecular weight between 1000 g/mole and 50,000 g/mole.

7. The composition of any one or more of embodiments 1-6, wherein the amphiphilic polymer has a molecular weight between 3,000 g/mole and 25,000 g/mole.

8. The composition of any one or more of embodiments 1-6, wherein the amphiphilic polymer has a molecular weight of at least 2000 g/mole.

9. The composition of any one or more of embodiments 1-8, wherein the amphiphilic polymer has a water surface tension of at least 50 dynes/cm² at a concentration of 0.1 wt %.

10. The composition of any one or more of embodiments 1-9, wherein the amphiphilic polymer is a copolymer of a hydrophilic block coupled with a hydrophobic block.

11. The composition of embodiment 10, wherein the hydrophobic block is selected from the group consisting of acrylates; methacrylates; acrylonitriles; methacrylonitrile; vinyls; aminoalkyls; styrenes; cellulose acetate phthalate; cellulose acetate succinate; hydroxypropylmethylcellulose phthalate; poly(D,L lactide); poly(D,L-lactide-co-glycolide); poly(D,L caprolactam); poly(D,L caprolactone); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers; hydrophobic peptide-based polymers and copolymers based on poly(L-amino acids); poly(ethylene-vinyl acetate) copolymers; silicone rubber; polyethylene; polypropylene; polydienes; maleic anyhydride copolymers of vinyl methylether and other vinyl ethers; polyamides; polyurethane; poly(ester urethanes); poly(ether urethanes); poly(ester-urea); poly(ethylenevinyl acetate); poly(D,L-lactic acid) oligomers; poly(D,L-lactic acid) polymers; poly(L-lactic acid) oligomers; poly(L-lactic acid) polymers; poly(glycolic acid); copolymers of lactic acid and glycolic acid; poly(caprolactone); poly(valerolactone); polyanhydrides; copolymers of poly(caprolactone); copolymers of poly(lactic acid); polystyrene; polyacrylates; butadienes; hydrophobic vitamins; carotenoids; retinols; cholecalciferol; calcitriol; hydroxycholecalciferol; ergocalciferol; alpha-tocopherol; alpha-tocopherol acetate; alpha-tocopherol nicotinate; and estradiol.

12. The composition of any one or more of embodiments 12-13, wherein the hydrophilic block is selected from the group consisting of carboxylic acids; polyoxyethylenes; poly ethylene oxide; polyacrylamides; polyacrylamide dimethylaminoethylmethacrylate copolymers; polyacrylamide diallyldimethylammonium chloride copolymers; polyacrylamide vinylbenzylthrimethylammonium chloride copolymers; polyacrylamide acrylic acid copolymers; polyacrylamide methacrylic acid copolymers; polyacrylamide 2-crrylamideo-2-methylpropane sulfonic acid copolymers; polyacrylamide styrene sulfonate copolymers; polyvincyl pyrrolidone; starches; starch derivatives; dextran; dextran derivatives; polypeptides; poly hyaluronic acids; alginic acids; polylactides; polyethyleneimines; polyionenes; polyacrylic acids; polyiminocarboxylates; gelatin; unsaturated ethylenic monocarboxylic acids; unsaturated ethylenic dicarboxylic acids; polybutyl acrylate; polybutyl methacrylate; and polycaprolactone.

13. The composition of any one or more of embodiments 10-12, wherein the hydrophobic block is a diblock repeat or a triblock repeat.

14. The composition of any one or more of embodiments 10-33, wherein the hydrophilic block is a diblock repeat or a triblock repeat.

15. The composition of any one or more of embodiments 1-14 further comprising one or more water-insoluble co-solutes with a partition coefficient of log P>3.0.

16. The composition of embodiment 15, wherein the one or more water-insoluble co-solutes are selected from the group consisting of hydrophobic vitamins; carotenoids; retinols; cholecalciferol; calcitriol; hydroxycholecalciferol; ergocalciferol; α-tocopherol; α-tocopherol acetate; α-tocopherol nicotinate; lipids; and estradiol, or a derivative of any of the foregoing.

17. The composition of any one or more of embodiments 1-16 further comprising a pharmaceutically acceptable carrier or excipient.

18. The composition of embodiment 17, wherein the pharmaceutically acceptable carrier or excipient is selected from the group consisting of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.), and phosphate buffered saline (PBS) or a pharmaceutically acceptable salt of any of the foregoing.

19. A method of preparing a water-insoluble steroid encapsulated in a nanoparticle form comprising:
dissolving at least one amphiphilic polymer and at least one water-insoluble steroid in an organic solvent to form an organic solvent stream; and
rapidly mixing the organic solvent stream with an aqueous stream.

20. The method of embodiment 19, wherein the one or more water-soluble steroids are selected from the group consisting of cholesterol; β-sitosterol; campesterol; ergosterol; cholecalciferol; fusidic acid; lanosterol; stigmasterol; cholestane; cyproterone; danazol; dydrogesterone; megestrol; canrenone; medrogestone; ethisterone; spironolactone; testosterone; estradiol; pregnenolone; 17-hydroxypregnenolone; progesterone; 17-hydroxyprogesterone; androstenedione; androsterone; epiandrosterone; dehydroepiandrosterone; epitestosterone; dihydrotestosterone; estrone; deoxycorticosterone; tixocortol pivalate; mometasone; aclometasone; betamethasone valerate; prednicarbate; clobetasol; ciclesonide; rimexolone; clobetasone; and halobetasol propionate; or a derivative of any of the foregoing.

21. The method of any one or more of embodiments 19-20, wherein the step of dissolving further comprises dissolving at least one water-insoluble co-solute in the organic solvent.

22. The method of embodiment 21, wherein the one or more water-insoluble co-solutes are selected from the group consisting of hydrophobic vitamins; carotenoids; retinols; cholecalciferol; calcitriol; hydroxycholecalciferol; ergocalciferol; α-tocopherol; α-tocopherol acetate; α-tocopherol nicotinate; and estradiol.

23. The method of any one or more of embodiments 19-22, wherein the amphiphilic polymer is a graft amphiphilic polymer, a block amphiphilic polymer, or a random amphiphilic polymer.

24. The method of any one or more of embodiments 19-23, wherein the amphiphilic polymer has a molecular weight between 1000 g/mole and 50,000 g/mole.

25. The method of any one or more of embodiments 19-24, wherein the amphiphilic polymer has a molecular weight between 3,000 g/mole and 25,000 g/mole.

26. The method of any one or more of embodiments 19-23, wherein the amphiphilic polymer has a molecular weight of at least 2000 g/mole.

27. The method of any one or more of embodiments 19-26, wherein the amphiphilic polymer has a water surface tension of at least 50 dynes/cm² at a concentration of 0.1 wt %.

28. The method of any one or more of embodiments 19-27, wherein the amphiphilic polymer is a copolymer of a hydrophilic block coupled with a hydrophobic block.

29. The method of embodiment 28, wherein the hydrophobic block is selected from the group consisting of acrylates; methacrylates; acrylonitriles; methacrylonitrile; vinyls; aminoalkyls; styrenes; cellulose acetate phthalate; cellulose acetate succinate; hydroxypropylmethylcellulose phthalate; poly(D,L lactide); poly(D,L-lactide-co-glycolide); poly(D,L caprolactam); poly(D,L caprolactone); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers; hydrophobic peptide-based polymers and copolymers based on poly(L-amino acids); poly(ethylene-vinyl acetate) (“EVA”) copolymers; silicone rubber; polyethylene; polypropylene; polydienes; maleic anyhydride copolymers of vinyl methylether and other vinyl ethers; polyamides; polyurethane; poly(ester urethanes); poly(ether urethanes); poly(ester-urea); poly(ethylenevinyl acetate); poly(D,L-lactic acid) oligomers; poly(D,L-lactic acid) polymers; poly(L-lactic acid) oligomers; poly(L-lactic acid) polymers; poly(glycolic acid); copolymers of lactic acid and glycolic acid; poly(caprolactone); poly (valerolactone); polyanhydrides; copolymers of poly(caprolactone); copolymers of poly(lactic acid); polystyrene; polyacrylates; butadienes; hydrophobic vitamins; carotenoids; retinols; cholecalciferol; calcitriol; hydroxycholecalciferol; ergocalciferol; alpha-tocopherol; alpha-tocopherol acetate; alpha-tocopherol nicotinate; and estradiol.

30. The method of embodiment 28, wherein the hydrophilic block is selected from the group consisting of carboxylic acids; polyoxyethylenes; poly ethylene oxide; polyacrylamides; polyacrylamide dimethylaminoethylmethacrylate copolymers; polyacrylamide diallyldimethylammonium chloride copolymers; polyacrylamide vinylbenzylthrimethylammonium chloride copolymers; polyacrylamide acrylic acid copolymers; polyacrylamide methacrylic acid copolymers; polyacrylamide 2-crrylamideo-2-methylpropane sulfonic acid copolymers; polyacrylamide styrene sulfonate copolymers; polyvincyl pyrrolidone; starches; starch derivatives; dextran; dextran derivatives; polypeptides; poly hyaluronic acids; alginic acids; polylactides; polyethyleneimines; polyionenes; polyacrylic acids; polyiminocarboxylates; gelatin; unsaturated ethylenic monocarboxylic acids; unsaturated ethylenic dicarboxylic acids; polybutyl acrylate; polybutyl methacrylate; and polycaprolactone.

31. The method of embodiment 30, wherein the hydrophobic block is selected from the group consisting of acrylates; methacrylates; acrylonitriles; methacrylonitrile; vinyls; aminoalkyls; styrenes; cellulose acetate phthalate; cellulose acetate succinate; hydroxypropylmethylcellulose phthalate; poly(D,L lactide); poly(D,L-lactide-co-glycolide); poly(D,L caprolactam); poly(D,L caprolactone); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers; hydrophobic peptide-based polymers and copolymers based on poly(L-amino acids); poly(ethylene-vinyl acetate) (“EVA”) copolymers; silicone rubber; polyethylene; polypropylene; polydienes; maleic anyhydride copolymers of vinyl methylether and other vinyl ethers; polyamides; polyurethane; poly(ester urethanes); poly(ether urethanes); poly(ester-urea); poly(ethylenevinyl acetate); poly(D,L-lactic acid) oligomers; poly(D,L-lactic acid) polymers; poly(L-lactic acid) oligomers; poly(L-lactic acid) polymers; poly(glycolic acid); copolymers of lactic acid and glycolic acid; poly(caprolactone); poly(valerolactone); polyanhydrides; copolymers of poly(caprolactone); copolymers of poly(lactic acid); polystyrene; polyacrylates; butadienes; hydrophobic vitamins; carotenoids; retinols; cholecalciferol; calcitriol; hydroxycholecalciferol; ergocalciferol; alpha-tocopherol; alpha-tocopherol acetate; alpha-tocopherol nicotinate; and estradiol.

32. The method of embodiment 28, wherein the hydrophobic block is a diblock repeat or a triblock repeat.

33. The method of embodiment 28, wherein the hydrophilic block is a diblock repeat or a triblock repeat.

34. The method of any one or more of embodiments 19-33, wherein the organic solvent includes at least one solvent selected from the group consisting of an alcohol, an ether, methanol, tetrahydrofuran, dimethylsulfoxide, dimethylformamide, and n-methylpyrollidone.

35. The method of any one or more of embodiments 19-34, wherein the aqueous stream includes deionized water.

36. The method of embodiment 35, wherein the aqueous stream includes at least one pharmaceutically acceptable carrier or excipient.

37. The method of any one or more of embodiments 19-36, wherein the ratio of water insoluble steroid to amphiphilic polymer in the organic solvent stream is 1:0.1 up to 1:10, and the water insoluble steroid is at a concentration of 0.01 mg/ml to 200 mg/ml.

38. The method of embodiment 37, wherein the water insoluble steroid is at a concentration of 1 mg/ml to 20 mg/ml in the organic solvent stream.

39. The method of embodiment 37, wherein the water insoluble steroid is at concentration in a range between and including any two values in 0.01 mg/ml steps from 0.01 mg/ml up to 200 mg/ml in the organic solvent stream.

40. A composition made by the method of any one or more of embodiments 19-39.

41. A method of treating disease comprising delivering to a patient in need thereof a composition of any one or more of embodiments 1-18, 40, and 42-47.

42. A composition of any one or more of the preceding embodiments wherein the water insoluble steroid is present in the nanoparticles at a concentration of 12.5% by weight or greater, where the percent is the weight of steroid divided by the total weight of nanoparticles.

43. A composition of any one or more of the preceding embodiments, wherein the water insoluble steroid is present in the nanoparticles is at a concentration of 12.5%-60% by weight, where the percent is the weight of steroid divided by the total weight of nanoparticles.

44. A composition of any one or more of the preceding embodiments, wherein the water insoluble steroid is present in the nanoparticles at a concentration in a range between and including any two integer values from 13%-60% by weight, where the percent is the weight of steroid divided by the total weight of nanoparticles.

45. A composition of any one or more of the preceding embodiments, wherein the water insoluble steroid is present in the nanoparticles at a concentration of 45% by weight, where the percent is the weight of steroid divided by the total weight of nanoparticles.

46. A composition of any one or more of the preceding embodiments, wherein the water insoluble steroid is present in the nanoparticles at a concentration such that an aqueous dispersion of the nanoparticles may be produced in an injectable form with steroid concentrations of 3 mg/ml to 200 mg/ml of active steroid.

47. A composition of any one or more of the preceding embodiments, wherein the water insoluble steroid is present in the nanoparticles at a concentration such that an aqueous dispersion of the nanoparticles may be produced in an injectable form with a steroid concentration in a range between and including any two integers from 3 mg/ml to 200 mg/ml.

Further embodiments herein may be formed by supplementing an embodiment with one or more element from any one or more other embodiment herein, and/or substituting one or more element from one embodiment with one or more element from one or more other embodiment herein.

### Examples

The following non-limiting examples are provided to illustrate particular embodiments. The embodiments throughout may be supplemented with one or more detail from one or more example below, and/or one or more element from an embodiment may be substituted with one or more detail from one or more example below.

### Example 1

### Progesterone Suspensions

A 2.5% (w/w) solution of α-tocopherol polyethylene glycol succinate 1000 was made using MilliQ water.

Progesterone was added to different aliquots at 20 and 30 mg/mL and both vortexed and sonicated for several minutes. The resulting suspensions were very cloudy and some settling was observed. Each sample was then shaken up again and was left so that some of the water would evaporate in hopes of forming an emulsion. However, significant precipitation of the progesterone was visible.

A 2.5% (w/w) solution of hydroxypropyl methylcellulose was made using MilliQ water. To an aliquot of this solution was added 0.5% (w/w) sodium lauryl sulfate. Both solutions were left to stir for at least half an hour.

Progesterone was added at 2.5% (w/w) to both solutions. Again, the solutions were left to stir for at least half an hour. No visible precipitate was present initially. However, in attempting to measure the size of the suspended particles using dynamic light scattering (DLS), no meaningful signal was measured as the particles are micron-sized. After three days of storage at 5° C., there was visible settling.

Since progesterone cannot be suspended in water in bulk form at high concentrations, in the nano-sized range, and in a stable formulation, other techniques are necessary.

### Example 2

### Progesterone Nanoparticles

Progesterone (22 mg) was added to 1 mL tetrahydrofuran (THF).

This solution was precipitated against 1 mL MilliQ water into a 19 mL reservoir of stirring MilliQ water using a CIJ mixer. The resulting suspension was very turbid and macroscopic crystals of progesterone were visible. Approximately half an hour after Flash Nano Precipitation (FNP), most of the solids had settled to the bottom.

Flash Nano Precipitation of progesterone without a stabilizer does not result in stable particles.

### Example 3

### Progesterone Loaded into PEG(5000)-b-PCL(5000) Nanoparticles

In attempting to adapt the solvent evaporation method of Eisenberg et al. for producing steroid-loaded polymeric micelles to FNP, the following example illustrates the limitations of the formulation in regards to applicability to all steroids.

Progesterone (120 mg) was added to 0.2 mL THF, as well as 60 mg of poly(ethylene glycol)(5000)-block-poly(caprolactone)(5000) (PEG-b-PCL). This formed a thick slurry and as much as possible was used.

This slurry was precipitated against 1 mL MilliQ water into a 5 mL reservoir of stirring MilliQ water using a CIJ mixer. The resulting suspension was very turbid and macroscopic aggregates were present. DLS measurements showed a polydisperse distribution, with micelles, 259 nm particles, and micron-sized aggregates.

The suspension was filtered using a PVDF 0.45 μm syringe filter and the THF was removed from the suspension using a rotary evaporator, leaving some precipitate behind. While subsequent DLS measurements did not show significantly different distributions, no more visible aggregates were present. The resulting suspension was assayed for progesterone concentrations, yielding an encapsulation efficiency of 1.4%.

While PCL may help to encapsulate other steroids such as β-estradiol, it does not efficiently encapsulate progesterone.

### Example 4

### Progesterone Loaded into PEG(5000)-b-PLA(3700) Nanoparticles

Progesterone (10 mg) was added to 1 mL THF, as well as 10 mg of poly(ethylene glycol)(5000)-block-poly(D,L-lactic acid) (3700) (PEG-b-PLA).

This solution was precipitated against 1 mL MilliQ water into a 9 mL reservoir of stirring MilliQ water using a CIJ mixer. The resulting suspension was very turbid and phase separation of the progesterone was apparent. DLS measurements of the supernatant showed the presence of micelles and particles with a diameter of 214 nm. While the progesterone encapsulation efficiency was not determined for this case, Example 9 exhibited a 9.5% encapsulation efficiency when using an organic stream with 10 times the concentration of both components.

If nanoparticles are to be used to suspend progesterone, a co-solute may be added to the core of the particles in order to keep the steroid in the core.

### Example 5

### Progesterone Loaded with Co-Solutes into PEG-b-PLA Nanoparticles

Progesterone (10 mg) was added to 1 mL THF, as well as 20 mg of PEG(5000)-b-PLA(3700) and 10 mg of one of following compounds:
(a) cholesterol
(b) bis(2-(17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl) 2,2′-oxydiacetate (subsequently referred to as prednisone diglycolate dimer)
(c) 2-(17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 2-(2-(icosyloxy)-2-oxoethoxy)acetate (subsequently referred to as prednisone cosanyl diglycolate)
(d) α-tocopherol.

Each solution was precipitated against 1 mL MilliQ water into a 9 mL reservoir of stirring MilliQ water using a CIJ mixer.

(a) The resulting suspension was opalescent and visually homogeneous. However, DLS measurements showed a bimodal distribution, with both micelles and large nanoparticles present.

The THF was removed from the suspension through a rotary evaporator. In the process, crystallization occurred, as seen by crystalline residues in the suspension. DLS measurements showed a greater amount of smaller particles after THF removal, as the crystals settled (FIG. 1).

Because of the visible precipitation, cholesterol may not be as suitable a co-solute for the formation of stable progesterone nanoparticles.

(b) The resulting suspension was very cloudy and macroscopic aggregates were present. Although the visible aggregates did settle over time, the suspension remained cloudy.

The suspension was then filtered through a PVDF 0.45 μm syringe filter and DLS measurements were done. The small size of the remaining particles indicates that they are most likely micelles and if they are loaded with progesterone and the prednisone dimer, it most likely is at a small loading (FIG. 2).

This formulation is not favorable for drug delivery purposes because of the poor encapsulation efficiency of drug.

(c) The resulting suspension was slightly cloudy, but opalescent. DLS measurements showed a bimodal distribution of particle sizes.

The suspension was dialyzed in a regenerated cellulose dialysis bag of MWCO 6-8 kDa against a bath of MilliQ water for 1 day. The bath was refreshed at least four times during the first hours. However, after the first hour of dialysis, there was flaky white precipitate corresponding to the progesterone, as prednisone cosanyl diglycolate has been shown to be stable when encapsulated in nanoparticles. Although DLS measurements show a stable size after dialysis, the particles most likely have very little, if any, progesterone in the cores (FIG. 3).

Effectively, this shows that similar steroids are not useful for stabilizing progesterone within polymeric nanoparticles.

(d) The resulting suspension was clear and opalescent. DLS measurements showed one population, with a size of 62 nm.

The THF was removed from the suspension using a rotary evaporator. No visible precipitate formed during the solvent removal. Further DLS measurements show insignificant change in size.

The next day, a small amount of crystalline residue was present at the bottom of the sample. However, DLS measurements did not show a decrease in particle size (FIG. 4A).

The same formulation was made using an equivalent amount of block copolymer (10 mg) and the results were similar, except the size was slightly larger (84 nm) (FIG. 4B).

This formulation is relatively stable and is able to encapsulate the progesterone for a sufficiently long enough period that the particles can be lyophilized.

### Example 6

### Lyophilization and Redispersion of Progesterone-Tocopherol Co-Loaded PEG-b-PLA Nanoparticles

Progesterone (40 mg) was added to 1 mL THF, as well as 40 mg of PEG(5000)-b-PLA(3700) and α-tocopherol.

This solution was precipitated using a MIVM, where the THF solution was pumped at 12 mL/min against three separate MilliQ water streams, each being pumped at 36 mL/min, for a 9:1 water:THF ratio. The resulting suspension was opalescent. DLS measurements gave the particle size as 149 nm.

Duplicate aliquots of 2 mL of the suspension were lyophilized with Pluronic F68 at the following cryoprotectant to nanoparticle weight ratios: 0:1, 5:1, 10:1, and 15:1. Each sample was then rehydrated with 2 mL MilliQ water and agitated by hand for 1 minute. DLS measurements were then taken. Those samples that were freeze-dried without Pluronic aggregated to form particles 3.2 times bigger than the original batch. For the samples with Pluronic, as the ratio of cryoprotectant to nanoparticles increased, the redispersed size went up from 1.6 times the original size (5:1) to 2.6 times (15:1) and the relative amounts of micelles also increased (FIG. 5).

Thus, when lyophilizing progesterone-tocopherol co-loaded PEG-b-PLA nanoparticles, Pluronic F68 can help to redisperse the particles to smaller sizes than if no cryoprotectant is used, but the amount should be below five times the weight of the nanoparticles.

### Example 7

### Quantification of Progesterone Concentration in Suspensions of Progesterone-Tocopherol Co-Loaded PEG-b-PLA Nanoparticles

To quantify progesterone concentrations in nanoparticle suspensions, the nanoparticles were first freeze-dried to remove the water from the solids. The lyophilized material was then dissolved in THF and then analyzed with UV/visible spectroscopy. The following describes the calibration curve preparation.

Progesterone (5 mg) was added to 5 mL THF, as well as 5 mg of PEG(5000)-b-PLA(3700) and α-tocopherol each.

This stock solution was used to create dilute samples ranging from 5 to 20 μg/mL of each component. UV/visible absorbance measurements were taken at the various concentrations and a calibration curve was constructed, such that the concentrations of progesterone and α-tocopherol in solution can be determined by a UV absorbance measurement.

These measurements were compared to ITV absorbance spectra of a pure solution of progesterone in THF.

Progesterone (5 mg) was added to 5 mL THF.

This stock solution was used to create dilute samples ranging from 10 to 20 μg/mL of progesterone. UV/visible absorbance measurements were taken at the various concentrations and a calibration curve was constructed (FIG. 6A).

The peak with a maximum at λ≈230 nm corresponds to the presence of progesterone in solution (FIG. 6B).

### Example 8

### Production, Lyophilization, and Redispersion of Concentrated Progesterone-Tocopherol Co-Loaded PEG-b-PLA Nanoparticles

Progesterone (600 mg) was added to 3 mL THF, as well as 600 mg of poly(ethylene glycol)(5000)-block-poly(D,L-lactic acid) (3700) (PEG-b-PLA) and

α-tocopherol. The solution was sonicated until all components were dissolved.

Of this solution, 2.5 mL was precipitated against 2.5 mL MilliQ water into a 20 mL reservoir of stirring MilliQ water using a CIJ mixer. The resulting suspension was a very opaque milky white, but no there was no visual phase separation. DLS measurements showed essentially one population with a size of 297 nm, although there was some bigger microparticles present, which corresponds to progesterone that was not encapsulated. This formulation yields a 69.4% progesterone encapsulation efficiency, with progesterone loadings of 24 wt % in the nanoparticles and a progesterone suspension concentration of 9.8 mg/mL.

To 8 mL of the nanoparticle suspension, Pluronic F68 (320 mg) was added at roughly a 1:1 ratio of cryoprotectant to nanoparticles. Four 2 mL aliquots were freeze-dried (trials 44-47).

From the original nanoparticle suspension, 10 mL were filtered through a nylon 5 μm syringe filter. Then, Pluronic F68 (320 mg) was added to 8 mL of the nanoparticles at a 1:1 ratio of cryoprotectant to nanoparticles. Four 2 mL aliquots were freeze-dried (trials 51-54).

To the dry lyophilized samples, 2 mL MilliQ water was added and each vial was hand agitated for 1 minute. The particle size and progesterone concentration was measured.

The reconstituted suspensions were then filtered through nylon 5 μm syringe filters; there was no significant change in pressure when injecting the suspensions through the filter, indicating no formation of filter cakes. The particle size and progesterone concentration was measured again.

The filtered suspensions were then expressed through 25 gauge needles; the dispersion flowed out through the needle without any problem and there is no concern about the suspension being too viscous for injection. The particle size and progesterone concentration was measured again.

Table 1, below shows that the concentrations of progesterone at each step are reproducible among the eight different trials.

Furthermore, the particle size distributions of the needle-injected suspensions of all trials are very similar, yielding an average size of 309 nm (FIG. 7A).

For this formulation, there is no significant particle growth (FIG. 7B) or loss (the table below) from Flash Nano Precipitation to lyophilization to reconstitution and to expression through a 25-gauge needle.

TABLE 1

| | Progesterone Concentration (mg/mL) | | |
|---|---|---|---|
| | | Filtered | Expressed |
| Trial # | Redispersed | (5 μm) | (25 gauge) |
| 44 | 10.8 | 9.2 | 9.9 |
| 45 | 10.8 | 9.8 | 9.3 |
| 46 | 10.5 | 9.3 | 9.8 |
| 47 | 10.3 | 9.5 | 9.7 |
| 51 | 11.1 | 9.3 | 9.8 |
| 52 | 10.9 | 9.5 | 9.7 |
| 53 | 10.8 | 9.9 | 8.8 |
| 54 | 10.6 | 9.0 | 9.5 |

### Example 9

### Production of Concentrated Progesterone-Loaded PEG(5000)-b-PLA(3700) Nanoparticles

Progesterone (400 mg) was added to 2 mL THF, as well as 400 mg of poly(ethylene glycol)(5000)-block-poly(D,L-lactic acid) (3700) (PEG-b-PLA). The solution was sonicated until all components were dissolved.

Of this solution, 2 mL was precipitated against 2 mL MilliQ water into a 16 mL reservoir of stirring MilliQ water using a CIJ mixer. The resulting suspension was very turbid and macroscopic aggregates were present. DLS measurements did not yield any meaningful data due to the presence of the large microparticles.

The nanoparticle suspension was filtered through nylon 5 μm syringe filters; more than one filter was used because when resistance was felt in injecting the suspension through the first one, a new filter was used. The filtrate had no visual precipitate and the suspension was somewhat opalescent. Particle size measurements revealed only one population, consisting of 37 nm micelles. This formulation yielded a 9.5% progesterone encapsulation efficiency, which is about seven times less than that which is achieved when α-tocopherol is added as a co-solvent.

Thus, α-tocopherol is critical in trapping the progesterone within the nanoparticle cores.

### Example 10

Comparison of Formulation to Other Formulations

Various solutions of progesterone, α-tocopherol, and poly(ethylene glycol)(5000)-block-poly(D,L-lactic acid) (3700) (PEG-b-PLA) were prepared and dissolved in 1.5 mL THF, as listed in Table 2 below. Trial 1 is Example 9 and trial 3 is Example 8.

TABLE 2

| | Pro- | α- | PEG- | | |
|---|---|---|---|---|---|
| | gesterone | Tocopherol | b-PLA | Diameter | Encapsulation |
| Trial | (mg) | (mg) | (mg) | (nm) | Efficiency (%) |
| 1 | 300 | 0 | 300 | 37 | 9.5 |
| 2 | 300 | 150 | 300 | 243 | 45.0 |
| 3 | 300 | 300 | 300 | 319 | 69.4 |
| 4 | 300 | 450 | 300 | 410 | 74.3 |
| 5 | 300 | 600 | 300 | too | 71.2 |
| | | | | polydisperse | |
| 6 | 15 | 15 | 15 | 94 | 42.8 |
| 7 | 75 | 75 | 75 | 120 | 54.8 |
| 8 | 150 | 150 | 150 | 195 | 67.7 |
| 9 | 300 | 300 | 300 | 270 | 69.4 |

For each trial, 1 mL of the solution was precipitated against 1 mL MilliQ water into a 8 mL reservoir of stirring MilliQ water using a CIJ mixer.

For trials 1-5, the suspensions were filtered through nylon 5 μm syringe filters and then characterized. For trials 6-9, the THF was removed from each suspension using a rotary evaporator and the suspensions were then filtered through nylon 5 μm syringe filters.

For each formulation, the particle size and concentration was measured after filtration. Encapsulation efficiencies were calculated based on the initial progesterone concentrations in the solutions used for mixing.

As is shown in the case of trials 1-5, the progesterone encapsulation efficiency increases as the ratio of α-tocopherol to progesterone is increased up to the 1:1 ratio, where higher ratios do not result in higher encapsulation (FIG. 8B). As has been shown in Example 9, the presence of α-tocopherol in the nanoparticle core is crucial for encapsulation of progesterone. Furthermore, as α-tocopherol concentrations increase, the nanoparticle sizes also increase, up to the point where the particle distributions are no longer monomodal and have micron-sized particles (FIG. 8A).

When comparing trials 5-7 and 2, increasing the concentrations of each component, while maintaining a 1:1:1 ratio of progesterone to α-tocopherol to PEG-b-PLA, results in an increased encapsulation efficiency up to approximately 100 mg of component per 1 mL of THF (FIG. 9B). The particle sizes also increase with rising concentrations (FIG. 9A).

However, in terms of encapsulation efficiencies, the formulation presented in Example 8 is the optimal, as the highest concentration of progesterone is achieved, while having the least loss of drug.

### Example 11

PEG(5000)-b-PLA(3700) (962 mg) was added to 3 mL THF as well as 535 mg of both progesterone and α-tocopherol. The solution was vortexed and sonicated until all solids were dissolved.

This solution was precipitated using a MIVM, where the THF solution was pumped at 8.6 mL/min against three separate cold aqueous Pluronic F68 (38.4 mg/mL) streams, each being pumped at 43 mL/min, for a 15:1 water:THF ratio. The resulting suspension was opalescent. DLS measurements gave the particle size as 354 nm. Aliquots of 3 mL of the suspension were lyophilized.

A pharmacokinetics study was conducted in Harlan Sprague Dawley male rats to determine pharmacokinetic parameters of the nanoparticle formulation. The lyophilized samples were reconstituted to an approximate progesterone concentration of 10 mg/mL. The formulation was tested intravenously (i.v.) and intramuscularly (i.m.), using four animals in each set. The i.v. administration was done at 1 mg/mL using 2 mL/kg and blood samples were taken at 0.08, 0.25, 0.5, 1, 2, 4, 6, 8 hours. Similarly, the i.m. administration was done at 10 mg/mL using 1 mL/kg with blood samples being taken at 0.25, 0.5, 1, 2, 4, 6, 8 hours. The blood was assayed for progesterone levels in the plasma. The data is shown in FIG. 10.

The pharmacokinetic parameters are presented in Table 3, below. The nanoparticulate formulation provides a vehicle for progesterone that yields high bioavailability of 47%.

TABLE 3

| | | | | AUC (0-t) | AUC (0-∞) |
|---|---|---|---|---|---|
| | Tₘₐₓ | Cₘₐₓ | T_{1/2} | (hr- | (hr- |
| Dose | (hr) | (ng/mL) | (hr) | ng/mL) | ng/mL) |
| i.v. 2 mg/kg | 0.08 | 794 | 1.6 | 287 | 321 |
| i.m. 10 mg/kg | 0.56 | 442 | 0.94 | 583 | 755 |

Bioavailability (F) 47%

### REFERENCES

(1) A. D. Woolfson, G. R. E. Elliot, C. A. Gilligan, C. M. Passmore. Design of an intravaginal ring for the controlled delivery of 17β-estradiol as its 3-acetate ester. J Control Release 1999, 61, 319-328.
(2) P. L. Soo, J. Lovric, P. Davidson, D. Maysinger, A. Eisenberg. Polycaprolactone-block-poly(ethylene oxide) Micelles: A Nanodelivery System for 17β-Estradiol. Mol. Pharmaceutics. 2005, 2(6), 519-527.
(3) H. F. Salem. Sustained-release progesterone nanosuspension following intramuscular injection in ovariectomized rats. Int. J. Nanomed 2010, 5, 943-954.
(4) D. Duchêne, D. Wouessidjewe, G. Ponchel. Cyclodextrins and carrier systems. J. Control Release 1999, 62, 263-268.
(5) R. Cavalli, E. Peira, O. Caputo, M. R. Gasco. Solid lipid nanoparticles as carriers of hydrocortisone and progesterone complexes with β-cyclodextrins. Int. J. Pharm. 1999, 182, 56-69.
(6) E. Memi [symbol] o\check{g}lu, A. Bochot, M. Sen, D. Duchêne, A. A. Hincal. Non-surfactant nanospheres of progesterone inclusion complexes with amphiphilic β-cyclodextrins. Int. J. Pharm. 2003, 251, 143-153.
(7) J. Matsumoto, Y. Nakada, K. Sakurai, T. Nakamura, Y. Takahashi. Preparation of nanoparticles consisted of poly(L-lactide)-poly(ethylene glycol)-pol(L-lactide) and their evaluation in vitro. Int. J. Pharm. 1999, 185, 93-101.
(8) Lim Soo, P.; Lovric, J.; Davidson, P.; Maysinger, D.; Eisenberg, A., Polycaprolactone-block-poly(ethylene oxide) Micelles: A Nanodelivery System for 17β-Estradiol. Molecular Pharmaceutics 2005, 2, (6), 519-527.
(9) Cavalli, R.; Peira, E.; Caputo, O.; Gasco, M. R., Solid lipid nanoparticles as carriers of hydrocortisone and progesterone complexes with beta-cyclodextrins. Int J Pharm 1999, 182, (1), 59-69.
(10) Memisoglu, E.; Bochot, A.; Sen, M.; Duchene, D.; Hincal, A. A., Non-surfactant nanospheres of progesterone inclusion complexes with amphiphilic beta-cyclodextrins. Int J Pharm 2003, 251, (1-2), 143-53.
(11) Matsumoto, J.; Nakada, Y.; Sakurai, K.; Nakamura, T.; Takahashi, Y., Preparation of nanoparticles consisted of poly(L-lactide)-poly(ethylene glycol)-poly(L-lactide) and their evaluation in vitro. Int J Pharm 1999, 185, (1), 93-101.
(12) Liu, Y.; Cheng, C.; Lui, Y.; Prud'homme, R. K.; Fox, R. O., Mixing in a multi-inlet vortex mixer (MIVM) for flash nanoprecipitation. Chemical Engineering Science 2007, 63, (11), 2829-2842.
(13) Liu, Y.; Cheng, C.; Lui, Y.; Prud'homme, R. K.; Fox, R. O., Mixing in a Multi-Inlet Vortex Mixer (MIVM) for Flash Nanoprecipitation. Chemical Engineering Research (submitted) 2007.
(14) Johnson, B. K. Flash nanoprecipitation of organic actives via confined micromixing and block copolymer stabilization. PhD Thesis, Princeton University, Princeton, 2003.
(15) Liu, Y.; Kathan, K.; Saad, W.; Prud'homme, R. K., Ostwald Ripening of β-Carotene Nanoparticles. Physical Review Letters 2007, 98, (3), 036102.
(16) Johnson, B. K.; Prud'homme, R. K., Flash NanoPrecipitation of Organic Actives and Block Copolymers using a Confined Impinging Jets Mixer. Australian Journal of Chemistry 2003, 56, (10), 1021-1024.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

What is claimed is:

1. A composition comprising an amphiphilic polymer and one or more water-insoluble steroids with a partition coefficient of log P>3.0, wherein the amphiphilic polymer is in a nanoparticle form, the one or more water insoluble steroids are encapsulated in the nanoparticle form, and the nanoparticle form is dispersable in an aqueous medium.

2. The composition of claim 1, wherein the nanoparticle form is 40 to 1000 nm.

3. The composition of claim 1, wherein the nanoparticle form is 80 to 350 nm.

4. The composition of claim 1, wherein the one or more water-insoluble steroids are selected from the group consisting of cholesterol; β-sitosterol; campesterol; ergosterol; cholecalciferol; fusidic acid; lanosterol; stigmasterol; cholestane; cyproterone; danazol; dydrogesterone; megestrol; canrenone; medrogestone; ethisterone; spironolactone; testosterone; estradiol; pregnenolone; 17-hydroxypregnenolone; progesterone; 17-hydroxyprogesterone; androstenedione; androsterone; epiandrosterone; dehydroepiandrosterone; epitestosterone; dihydrotestosterone; estrone; deoxycorticosterone; tixocortol pivalate; mometasone; aclometasone; betamethasone valerate; prednicarbate; clobetasol; ciclesonide; rimexolone; clobetasone; and halobetasol propionate; or a derivative of any of the foregoing.

5. The composition of claim 1, wherein the amphiphilic polymer is a graft amphiphilic polymer, a block amphiphilic polymer, or a random amphiphilic polymer.

6. The composition of claim 1, wherein the amphiphilic polymer has a molecular weight between 1000 g/mole and 50,000 g/mole.

7. The composition of claim 1, wherein the amphiphilic polymer has a molecular weight between 3,000 g/mole and 25,000 g/mole.

8. The composition of claim 1, wherein the amphiphilic polymer has a molecular weight of at least 2000 g/mole.

9. The composition of claim 1, wherein the amphiphilic polymer has a water surface tension of at least 50 dynes/cm² at a concentration of 0.1 wt %.

10. The composition of claim 1, wherein the amphiphilic polymer is a copolymer of a hydrophilic block coupled with a hydrophobic block.

11. The composition of claim 10, wherein the hydrophobic block is selected from the group consisting of acrylates; methacrylates; acrylonitriles; methacrylonitrile; vinyls; aminoalkyls; styrenes; cellulose acetate phthalate; cellulose acetate succinate; hydroxypropylmethylcellulose phthalate; poly(D,L lactide); poly(D,L-lactide-co-glycolide); poly(D,L caprolactam); poly(D,L caprolactone); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers; hydrophobic peptide-based polymers and copolymers based on poly(L-amino acids); poly(ethylene-vinyl acetate) copolymers; silicone rubber; polyethylene; polypropylene; polydienes; maleic anyhydride copolymers of vinyl methylether and other vinyl ethers; polyamides; polyurethane; poly(ester urethanes); poly(ether urethanes); poly(ester-urea); poly(ethylenevinyl acetate); poly(D,L-lactic acid) oligomers; poly(D,L-lactic acid) polymers; poly(L-lactic acid) oligomers; poly(L-lactic acid) polymers; poly (glycolic acid); copolymers of lactic acid and glycolic acid; poly(caprolactone); poly(valerolactone); polyanhydrides; copolymers of poly(caprolactone); copolymers of poly(lactic acid); polystyrene; polyacrylates; butadienes; hydrophobic vitamins; carotenoids; retinols; cholecalciferol; calcitriol; hydroxycholecalciferol; ergocalciferol; alpha-tocopherol; alpha-tocopherol acetate; alpha-tocopherol nicotinate; and estradiol.

12. The composition of claim 10, wherein the hydrophilic block is selected from the group consisting of carboxylic acids; polyoxyethylenes; poly ethylene oxide; polyacrylamides; polyacrylamide dimethylaminoethylmethacrylate copolymers; polyacrylamide diallyldimethylammonium chloride copolymers; polyacrylamide vinylbenzylthrimethylammonium chloride copolymers; polyacrylamide acrylic acid copolymers; polyacrylamide methacrylic acid copolymers; polyacrylamide 2-crrylamideo-2-methylpropane sulfonic acid copolymers; polyacrylamide styrene sulfonate copolymers; polyvincyl pyrrolidone; starches; starch derivatives; dextran; dextran derivatives; polypeptides; poly hyaluronic acids; alginic acids; polylactides; polyethyleneimines; polyionenes; polyacrylic acids; polyiminocarboxylates; gelatin; unsaturated ethylenic monocarboxylic acids; unsaturated ethylenic dicarboxylic acids; polybutyl acrylate; polybutyl methacrylate; and polycaprolactone.

13. The composition of claim 10, wherein the hydrophobic block is a diblock repeat or a triblock repeat.

14. The composition of claim 10, wherein the hydrophilic block is a diblock repeat or a triblock repeat.

15. The composition of claim 1 further comprising one or more water-insoluble co-solutes with a partition coefficient of log P>3.0.

16. The composition of claim 15, wherein the one or more water-insoluble co-solutes are selected from the group consisting of hydrophobic vitamins; carotenoids; retinols; cholecalciferol; calcitriol; hydroxycholecalciferol; ergocalciferol; α-tocopherol; α-tocopherol acetate; α-tocopherol nicotinate; lipids; and estradiol, or a derivative of any of the foregoing.

17. The composition of claim 1 further comprising a pharmaceutically acceptable carrier or excipient.

18. The composition of claim 17, wherein the pharmaceutically acceptable carrier or excipient is selected from the group consisting of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.), and phosphate buffered saline (PBS) or a pharmaceutically acceptable salt of any of the foregoing.

19. A method of preparing a water-insoluble steroid encapsulated in a nanoparticle form comprising:
dissolving at least one amphiphilic polymer and at least one water-insoluble steroid in an organic solvent to form an organic solvent stream; and
rapidly mixing the organic solvent stream with an aqueous stream.

20. The method of claim 19, wherein the one or more water-soluble steroids are selected from the group consisting of cholesterol; β-sitosterol; campesterol; ergosterol; cholecalciferol; fusidic acid; lanosterol; stigmasterol; cholestane; cyproterone; danazol; dydrogesterone; megestrol; canrenone; medrogestone; ethisterone; spironolactone; testosterone; estradiol; pregnenolone; 17-hydroxypregnenolone; progesterone; 17-hydroxyprogesterone; androstenedione; androsterone; epiandrosterone; dehydroepiandrosterone; epitestosterone; dihydrotestosterone; estrone; deoxycorticosterone; tixocortol pivalate; mometasone; aclometasone; betamethasone valerate; prednicarbate; clobetasol; ciclesonide; rimexolone; clobetasone; and halobetasol propionate; or a derivative of any of the foregoing.

21. The method of claim 19, wherein the step of dissolving further comprises dissolving at least one water-insoluble co-solute in the organic solvent.

22. The method of claim 21, wherein the one or more water-insoluble co-solutes are selected from the group consisting of hydrophobic vitamins; carotenoids; retinols; cholecalciferol; calcitriol; hydroxycholecalciferol; ergocalciferol; α-tocopherol; α-tocopherol acetate; α-tocopherol nicotinate; and estradiol.

23. The method of claim 19, wherein the amphiphilic polymer is a graft amphiphilic polymer, a block amphiphilic polymer, or a random amphiphilic polymer.

24. The method of claim 19, wherein the amphiphilic polymer has a molecular weight between 1000 g/mole and 50,000 g/mole.

25. The method of claim 19, wherein the amphiphilic polymer has a molecular weight between 3,000 g/mole and 25,000 g/mole.

26. The method of claim 19, wherein the amphiphilic polymer has a molecular weight of at least 2000 g/mole.

27. The method of claim 19, wherein the amphiphilic polymer has a water surface tension of at least 50 dynes/cm² at a concentration of 0.1 wt %.

28. The method of claim 19, wherein the amphiphilic polymer is a copolymer of a hydrophilic block coupled with a hydrophobic block.

29. The method of claim 28, wherein the hydrophobic block is selected from the group consisting of acrylates; methacrylates; acrylonitriles; methacrylonitrile; vinyls; aminoalkyls; styrenes; cellulose acetate phthalate; cellulose acetate succinate; hydroxypropylmethylcellulose phthalate; poly(D,L lactide); poly(D,L-lactide-co-glycolide); poly(D,L caprolactam); poly(D,L caprolactone); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers; hydrophobic peptide-based polymers and copolymers based on poly(L-amino acids); poly(ethylene-vinyl acetate) copolymers; silicone rubber; polyethylene; polypropylene; polydienes; maleic anyhydride copolymers of vinyl methylether and other vinyl ethers; polyamides; polyurethane; poly(ester urethanes); poly(ether urethanes); poly(ester-urea); poly(ethylenevinyl acetate); poly(D,L-lactic acid) oligomers; poly(D,L-lactic acid) polymers; poly(L-lactic acid) oligomers; poly(L-lactic acid) polymers; poly(glycolic acid); copolymers of lactic acid and glycolic acid; poly(caprolactone); poly(valerolactone); polyanhydrides; copolymers of poly(caprolactone); copolymers of poly(lactic acid); polystyrene; polyacrylates; butadienes; hydrophobic vitamins; carotenoids; retinols; cholecalciferol; calcitriol; hydroxycholecalciferol; ergocalciferol; alpha-tocopherol; alpha-tocopherol acetate; alpha-tocopherol nicotinate; and estradiol.

30. The method of claim 28, wherein the hydrophilic block is selected from the group consisting of carboxylic acids; polyoxyethylenes; poly ethylene oxide; polyacrylamides; polyacrylamide dimethylaminoethylmethacrylate copolymers; polyacrylamide diallyldimethylammonium chloride copolymers; polyacrylamide vinylbenzylthrimethylammonium chloride copolymers; polyacrylamide acrylic acid copolymers; polyacrylamide methacrylic acid copolymers; polyacrylamide 2-crrylamideo-2-methylpropane sulfonic acid copolymers; polyacrylamide styrene sulfonate copolymers; polyvincyl pyrrolidone; starches; starch derivatives; dextran; dextran derivatives; polypeptides; poly hyaluronic acids; alginic acids; polylactides; polyethyleneimines; polyionenes; polyacrylic acids; polyiminocarboxylates; gelatin; unsaturated ethylenic monocarboxylic acids; unsaturated ethylenic dicarboxylic acids; polybutyl acrylate; polybutyl methacrylate; and polycaprolactone.

31. The method of claim 30, wherein the hydrophobic block is selected from the group consisting of acrylates; methacrylates; acrylonitriles; methacrylonitrile; vinyls; aminoalkyls; styrenes; cellulose acetate phthalate; cellulose acetate succinate; hydroxypropylmethylcellulose phthalate; poly(D,L lactide); poly(D,L-lactide-co-glycolide); poly(D,L caprolactam); poly(D,L caprolactone); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers; hydrophobic peptide-based polymers and copolymers based on poly(L-amino acids); poly(ethylene-vinyl acetate) copolymers; silicone rubber; polyethylene; polypropylene; polydienes; maleic anyhydride copolymers of vinyl methylether and other vinyl ethers; polyamides; polyurethane; poly(ester urethanes); poly(ether urethanes); poly(ester-urea); poly(ethylenevinyl acetate); poly(D,L-lactic acid) oligomers; poly(D,L-lactic acid) polymers; poly(L-lactic acid) oligomers; poly(L-lactic acid) polymers; poly(glycolic acid); copolymers of lactic acid and glycolic acid; poly(caprolactone); poly(valerolactone); polyanhydrides; copolymers of poly(caprolactone); copolymers of poly(lactic acid); polystyrene; polyacrylates; butadienes; hydrophobic vitamins; carotenoids; retinols; cholecalciferol; calcitriol; hydroxycholecalciferol; ergocalciferol; alpha-tocopherol; alpha-tocopherol acetate; alpha-tocopherol nicotinate; and estradiol.

32. The method of claim 28, wherein the hydrophobic block is a diblock repeat or a triblock repeat.

33. The method of claim 28, wherein the hydrophilic block is a diblock repeat or a triblock repeat.

34. The method of claim 19, wherein the organic solvent includes at least one solvent selected from the group consisting of an alcohol, an ether, methanol, tetrahydrofuran, dimethylsulfoxide, dimethylformamide, and n-methylpyrollidone.

35. The method of claim 19, wherein the aqueous stream includes deionized water.

36. The method of claim 35, wherein the aqueous stream includes at least one pharmaceutically acceptable carrier or excipient.

37. A composition made by the method of claim 19.

38. A method of treating disease comprising delivering to a patient in need thereof a composition comprising an amphiphilic polymer and one or more water-insoluble steroids with a partition coefficient of log P>3.0, wherein the amphiphilic polymer is in a nanoparticle form and the one or more water insoluble steroids are encapsulated in the nanoparticle form, and the nanoparticle form is dispersable in an aqueous medium.

39. The method of claim 38, wherein the nanoparticle form is 40 to 1000 nm.

40. The method of claim 38, wherein the nanoparticle form is 80 to 350 nm.

41. The method of claim 38, wherein the one or more water-insoluble steroids are selected from the group consisting of cholesterol; β-sitosterol; campesterol; ergosterol; cholecalciferol; fusidic acid; lanosterol; stigmasterol; cholestane; cyproterone; danazol; dydrogesterone; megestrol; canrenone; medrogestone; ethisterone; spironolactone; testosterone; estradiol; pregnenolone; 17-hydroxypregnenolone; progesterone; 17-hydroxyprogesterone; androstenedione; androsterone; epiandrosterone; dehydroepiandrosterone; epitestosterone; dihydrotestosterone; estrone; deoxycorticosterone; tixocortol pivalate; mometasone; aclometasone; betamethasone valerate; prednicarbate; clobetasol; ciclesonide; rimexolone; clobetasone; and halobetasol propionate; or a derivative of any of the foregoing.

42. The method of claim 38, wherein the amphiphilic polymer is a graft amphiphilic polymer, a block amphiphilic polymer or a random amphiphilic polymer.

43. The method of claim 38, wherein the amphiphilic polymer has a molecular weight between 1000 g/mole and 50,000 g/mole.

44. The method of claim 38, wherein the amphiphilic polymer has a molecular weight between 3,000 g/mole and 25,000 g/mole.

45. The method of claim 38, wherein the amphiphilic polymer has a molecular weight of at least 2000 g/mole.

46. The method of claim 38, wherein the amphiphilic polymer has a water surface tension of at least 50 dynes/cm² at a concentration of 0.1 wt %.

47. The method of claim 38, wherein the amphiphilic polymer is a copolymer of a hydrophilic block coupled with a hydrophobic block.

48. The method of claim 47, wherein the hydrophobic block is selected from the group consisting of acrylates; methacrylates; acrylonitriles; methacrylonitrile; vinyls; aminoalkyls; styrenes; cellulose acetate phthalate; cellulose acetate succinate; hydroxypropylmethylcellulose phthalate; poly(D,L lactide); poly(D,L-lactide-co-glycolide); poly(D,L caprolactam); poly(D,L caprolactone); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers; hydrophobic peptide-based polymers and copolymers based on poly(L-amino acids); poly(ethylene-vinyl acetate) copolymers; silicone rubber; polyethylene; polypropylene; polydienes; maleic anyhydride copolymers of vinyl methylether and other vinyl ethers; polyamides; polyurethane; poly(ester urethanes); poly(ether urethanes); poly(ester-urea); poly(ethylenevinyl acetate); poly(D,L-lactic acid) oligomers; poly(D,L-lactic acid) polymers; poly(L-lactic acid) oligomers; poly(L-lactic acid) polymers; poly(glycolic acid); copolymers of lactic acid and glycolic acid; poly(caprolactone); poly(valerolactone); polyanhydrides; copolymers of poly(caprolactone); copolymers of poly(lactic acid); polystyrene; polyacrylates; butadienes; hydrophobic vitamins; carotenoids; retinols; cholecalciferol; calcitriol; hydroxycholecalciferol; ergocalciferol; alpha-tocopherol; alpha-tocopherol acetate; alpha-tocopherol nicotinate; and estradiol.

49. The method of claim 47, wherein the hydrophilic block is selected from the group consisting of carboxylic acids; polyoxyethylenes; poly ethylene oxide; polyacrylamides; polyacrylamide dimethylaminoethylmethacrylate copolymers; polyacrylamide diallyldimethylammonium chloride copolymers; polyacrylamide vinylbenzylthrimethylammonium chloride copolymers; polyacrylamide acrylic acid copolymers; polyacrylamide methacrylic acid copolymers; polyacrylamide 2-crrylamideo-2-methylpropane sulfonic acid copolymers; polyacrylamide styrene sulfonate copolymers; polyvincyl pyrrolidone; starches; starch derivatives; dextran; dextran derivatives; polypeptides; poly hyaluronic acids; alginic acids; polylactides; polyethyleneimines; polyionenes; polyacrylic acids; polyiminocarboxylates; gelatin; unsaturated ethylenic monocarboxylic acids; unsaturated ethylenic dicarboxylic acids; polybutyl acrylate; polybutyl methacrylate; and polycaprolactone.

50. The method of claim 38, wherein the hydrophobic block is a diblock repeat or a triblock repeat.

51. The method of claim 38, wherein the hydrophilic block is a diblock repeat or a triblock repeat.

52. The method of claim 38, wherein dissolving includes dissolving one or more water-insoluble co-solutes with a partition coefficient of log P>3.0.

53. The method of claim 52, wherein the one or more water-insoluble co-solutes are selected from the group consisting of hydrophobic vitamins; carotenoids; retinols; cholecalciferol; calcitriol; hydroxycholecalciferol; ergocalciferol; α-tocopherol; α-tocopherol acetate; α-tocopherol nicotinate; lipids; and estradiol.
